(19) United States
(12) Patent Application Publication
(10) Pub. No.: US 2021/0196719 A1
(43) Pub. Date: Jul. 1, 2021

(54) USE OF CDK4/6 INHIBITOR IN COMBINATION WITH EGFR INHIBITOR IN THE PREPARATION OF MEDICAMENT FOR TREATING TUMOR DISEASES

(71) Applicant: JIANGSU HENGRUI MEDICINE CO., LTD., Jiangsu (CN)

(72) Inventors: Lei ZHANG, Jiangsu (CN); Changyong YANG, Jiangsu (CN); Cheng LIAO, Jiangsu (CN); Lianshan ZHANG, Jiangsu (CN)

(73) Assignee: JIANGSU HENGRUI MEDICINE CO., LTD., Jiangsu (CN)

(21) Appl. No.: 17/057,513

(22) PCT Filed: May 22, 2019
(86) PCT No.: PCT/CN2019/087945

§ 371 Date: Nov. 20, 2020

(30) Foreign Application Priority Data
May 23, 2018 (CN) 201810499596.2
Sep. 18, 2018 (CN) 201811086544.9
Oct. 11, 2018 (CN) 201811182296.8

(51) Int. Cl. A61K 31/519; A61K 31/506; A61P 35/00; A61K 31/5377

(52) U.S. Cl. CPC A61K 31/519; A61K 31/5377; A61P 35/00; A61K 31/506

(57) ABSTRACT

Provided in the present invention is a use of CDK4/6 inhibitor in combination with EGFR inhibitor in the preparation of a medicament for treating tumor diseases. In particular, provided in the invention is a use of a cyclin-dependent kinase 4 and 6 inhibitor (CDK4/6i) in combination with a human epidermal growth factor receptor inhibitor (EGFRi) for the preparation of a medicament for preventing or treating tumor diseases.

The present application claims the priority to Chinese Patent Application No. CN201810499596.2 filed on May 23, 2018, Chinese Patent Application No. CN201811086544.9 filed on Sep. 18, 2018, and Chinese Patent Application No. CN201811182296.8 filed on Oct. 11, 2018, the contents of which are incorporated herein by reference in their entirety.

### TECHNICAL FIELD

The invention pertains to the pharmaceutical field, which particularly relates to a use of cyclin-dependent kinase 4 and 6 (CDK4/6) inhibitor (CDK4/6i) in combination with human epidermal growth factor receptor inhibitor (EGFRi) in the manufacture of a medicament for preventing or treating non-small cell lung cancer (NSCLC).

### BACKGROUND ARTS

Lung cancer has become the leading cause of cancer deaths worldwide. In China, lung cancer ranks first in terms of cancer incidence and mortality. Although several newer generations of cytotoxic drugs and targeted therapies have been introduced in the past 20 years, patients with advanced lung cancer, especially those without known driver mutation genes, still have a poor survival prognosis. The advanced or metastatic lung cancer is still a fatal disease with a large number of unmet medical needs.

Non-small cell lung cancer (NSCLC) accounts for about 85% of all lung cancers. About 75% of NSCLC patients are already in the advanced stage when discovered, and the 5-year survival rate is quite low. There is still a great clinical need to choose an appropriate systemic treatment for the patients suffering from the advanced or metastatic NSCLC. NSCLC can be classified into squamous cell carcinoma and non-squamous cell carcinoma. Non-squamous cell carcinomas include adenocarcinoma, large cell carcinoma and other subtypes of cell carcinoma. Patients suffering from non-squamous cell carcinoma are further classified according to whether there is a driver mutation gene (EGFR mutation or ALK gene rearrangement).

EGFR (Epidermal Growth Factor Receptor) is a member of the erbB receptor family, which is transmembrane protein tyrosine kinase. By binding to its ligand, such as epidermal growth factor (EGF), EGFR can form a homodimer on the cell membrane or form a heterodimer with other receptors in the family, such as erbB2, erbB3, or erbB4. The formation of these dimers can cause the phosphorylation of key tyrosine residues in EGFR cells, thereby activating a number of downstream signaling pathways in cells. These intracellular signaling pathways play an important role in cell proliferation, survival and anti-apoptosis. Disorders of EGFR signal transduction pathways, including increased expression of ligands and receptors, EGFR gene amplification and mutation and the like, can promote malignant transformation of cells and play an important role in tumor cell proliferation, invasion, metastasis and angiogenesis. The over expression of EGFR has been reported in many human malignant diseases, including bladder cancer, brain tumors, head and neck cancer, pancreatic cancer, lung cancer, breast cancer, ovarian cancer, colon cancer, prostate cancer, and kidney cancer. In many cases, the over expression of EGFR is related to the poor prognosis of patients.

In addition, a large number of studies have found that tumors are related to cell cycle abnormalities. Most tumors have a large number of mutations of mitotic signaling protein/anti-mitotic signaling protein defects, genomic instability (GIN) and chromosome instability (CIN). These three basic cell cycle defects are directly or indirectly caused by the uncontrolled cyclin-dependent kinase (CDK). Cyclin B/CDK1, Cyclin A/CDK2, Cyclin E/CDK2, Cyclin D/CDK4, Cyclin D/CDK6 and other heterodimers (including CDK3 and CDK7) are important regulators of cell cycle progression. Some CDK inhibitors have been published, among which CDK4/6 inhibitors are abemaciclib, ribociclib, palbociclib, etc.

The prior art discloses a use of some CDK inhibitors in combination with EGFR kinase inhibitors in the manufacture of a medicament for treating non-small cell lung cancer, for example, the report “Synergistic combinations between the oral CDK inhibitor, seliciclib, and either EGFR inhibitors or DNA damaging agents in NSCLC” (AACR Annual Meeting—Apr 14-18, 2007; Los Angeles, Calif.) discloses that CDK inhibitor seliciclib in combination with EGFR kinase inhibitor has a synergistic effect in the treatment of NSCLC, wherein seliciclib is an inhibitor targeting CDK2, CDK7 and CDK9.

WO2017160568A discloses a use of necitumumab which is a recombinant human-derived IgG1 monoclonal antibody in combination with abemaciclib which is a cyclin-dependent kinase CDK4 and CDK6 inhibitor in the treatment of non-small cell lung cancer. References “PD 0332991, a selective cyclin D kinase 4/6 inhibitor, sensitizes lung cancer cells to treatment with epidermal growth factor receptor tyrosine kinase inhibitors” (Oncotarget. 2016 Dec. 20; 7(51): 84951-84964) discloses that PD 0332991 (i.e., palbociclib) and Gefitinib are used to inhibit the growth of lung adenocarcinoma cell lines.

WO2014183520 provides an effective CDK4/6 inhibitor, the structure of which is represented by formula (I), and WO2016124067 discloses the hydroxyethyl sulfonate of the novel CDK4/6 inhibitor

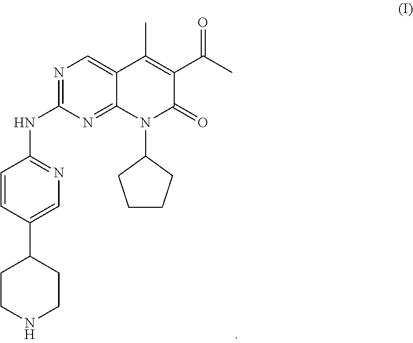 [SMILES: CC(=O)c1c(C)c2cnc(Nc3ccc(C4CCNCC4)cn3)nc2n(C2CCCC2)c1=O]

WO2016054987A discloses a 4-substituted-2-(N-(5-allylamido)phenyl)amino)pyrimidine derivative represented by formula (II), which has inhibitory activity against the EGFR-L858R mutant, the EGFR-T790M mutant and the mutant activated by exon 19 deletion, and can be used to treat diseases solely or partially mediated by the activity of EGFR mutants. WO2017161937A discloses the mesylate of the EGFR inhibitor represented by formula (II),

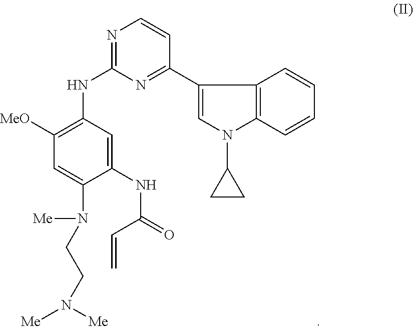 [SMILES: C=CC(=O)Nc1cc(Nc2nccc(-c3cn(C4CC4)c4ccccc34)n2)c(OC)cc1N(C)CCN(C)C]

The present invention provides a novel use of CDK4/6 inhibitor in combination with EGFR inhibitor in the manufacture of a medicament for preventing or treating non-small cell lung cancer, which exhibits good effects.

Content of the Present Invention

The invention provides a use of CDK4/6 inhibitor in combination with EGFR inhibitor in the manufacture of a medicament for preventing or treating tumor disease.

The tumor disease of the present invention can be selected from the group consisting of breast cancer, ovarian cancer, prostate cancer, melanoma, brain tumor, esophageal cancer, stomach cancer, liver cancer, pancreatic cancer, colorectal cancer, lung cancer, kidney cancer, skin cancer, glioblastoma, neuroblastoma, sarcoma, liposarcoma, osteochondroma, osteoma, osteosarcoma, seminoma, testicular tumor, uterine cancer, head and neck tumor, multiple myeloma, malignant lymphoma, polycythemia vera, leukemia, thyroid tumor, ureteral tumor, bladder tumor, gallbladder cancer, cholangiocarcinoma or choriocarcinoma; preferably, non-small cell lung cancer.

The invention provides a use of CDK4/6 inhibitor in combination with EGFR inhibitor in the manufacture of a medicament for preventing or treating tumor disease, wherein the tumor disease is an EGFR mutation tumor disease.

The EGFR mutation tumor disease of the present invention is preferably non-small cell lung cancer, and preferably, the EGFR mutant is L858R EGFR mutant and/or T790M EGFR mutant.

In the optional embodiments, the non-small cell lung cancer of the present invention is squamous cell carcinoma and non-squamous cell carcinoma, preferably, non-phosphorous cell carcinoma, wherein the non-phosphorous cell carcinoma can be adenocarcinoma, large cell carcinoma and other subtypes of cell carcinoma.

In the optional embodiments, the CDK4/6 inhibitor can be abemaciclib, ribociclib, palbociclib, alvocidib, trilaciclib, voruciclib, AT-7519, G1T-38, FLX-925, INOC-005, G1T28-1, BPI-1178, gossypin, G1 T30-1, GZ-38-1, P-276-00, staurosporine, R-547, PAN-1215, PD-0183812, AG-024322, NSC-625987, CGP-82996, PD-171851 or the compound represented by formula (I), the complex thereof or the pharmaceutically acceptable salt thereof, preferably abemaciclib, ribociclib, palbociclib, alvocidib or the compound represented by formula (I), the complex thereof or the pharmaceutically acceptable salt thereof, the most preferably the compound represented by formula (I), the complex thereof or the pharmaceutically acceptable salt thereof,

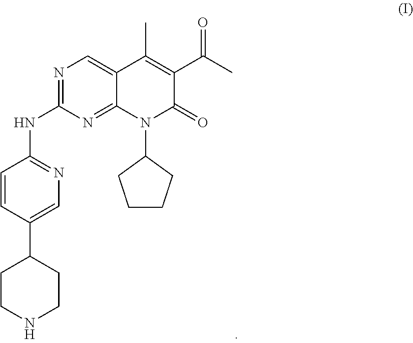 [SMILES: CC(=O)c1c(C)c2cnc(Nc3ccc(C4CCNCC4)cn3)nc2n(C2CCCC2)c1=O]

In the optional embodiments, the EGFR inhibitor can be osimertinib, gefitinib, erlotinib, olmutinib, icotinib, pyrotinib, vandetanib, brigatinib, dacomitinib, afatinib, neratinib, lapatinib, ABT-414, varlitinib, HLX-07, tesevatinib, theliatinib, epitinib succinate, S-222611, poziotinib, AST-2818, GNS-1480, mavelertinib, AP-32788, AZD-3759, nazartinib, Sym-013, allitinib tosylate, tarloxotinib bromide, CK-101, QL-1203, JNJ-61186372, SKLB-1028, TAS-121, Hemay-020, Hemay-022, NRC-2694-A, simotinib hydrochloride, SPH-1188-11, GR-1401, SYN-004, ABBV-221, MP-0274, GC-1118, BPI-15000, DBPR-112, Pirotinib, PB-357, lifirafenib, SCT-200, QLNC-120, agerafenib hydrochloride or the compound represented by formula (II), the stereoisomer thereof, the complex thereof or the pharmaceutically acceptable salt thereof, preferably olmutinib, afatinib, osimertinib, CK-101, erlotinib, icotinib, gefitinib or the compound represented by formula (II), the stereoisomer thereof, the complex thereof or the pharmaceutically acceptable salt thereof, the most preferably the compound represented by formula (II), the stereoisomer thereof, the complex thereof or the pharmaceutically acceptable salt thereof,

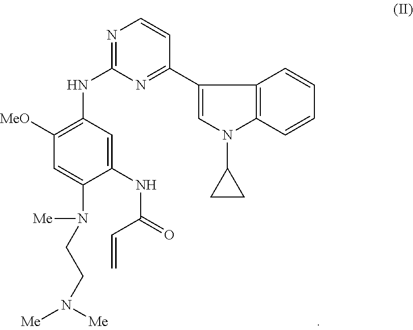 [SMILES: C=CC(=O)Nc1cc(Nc2nccc(-c3cn(C4CC4)c4ccccc34)n2)c(OC)cc1N(C)CCN(C)C]

The pharmaceutically acceptable salt of the present invention can be hydrochloride, phosphate, hydrogen phosphate, sulfate, hydrogen sulfate, sulfite, acetate, oxalate, malonate, valerate, glutamate, oleate, palmitate, stearate, laurate, borate, p-toluenesulfonate, mesylate, hydroxyethyl sulfonate, maleate, malate, tartrate, benzoate, pamoate, salicylate, vanillate, mandelate, succinate, gluconate, lactobionate or lauryl sulfonate, etc.

In the preferable embodiments, the pharmaceutically acceptable salt of the compound represented by represented by formula (I) is hydroxyethyl sulfonate.

In the preferable embodiments, the pharmaceutically acceptable salt of the compound represented by represented by formula (II) is mesylate.

In the use of CDK4/6 inhibitor in combination with EGFR inhibitor in the manufacture of a medicament for preventing or treating tumor disease, the frequency of administration of the CDK4/6 inhibitor can be once a day, twice a day, or three times a day, and the frequency of administration of the EGFR inhibitor can be once a day, twice a day, or three times a day.

In the optional embodiments, the dose of the CDK4/6 inhibitor is 1-1000 mg, and the frequency of administration thereof can be once a day, twice a day, or three times a day, and the dose of the EGFR inhibitor is 1-1000 mg, and the frequency of administration thereof can be once a day, twice a day, or three times a day.

The dose of the CDK4/6 inhibitor of the present invention can be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, 125 mg, 150 mg, 175 mg, 200 mg, 250 mg, 300 mg, 350 mg, 400 mg, 450 mg, 500 mg, 600 mg, 700 mg, 750 mg, 800 mg, 900 mg or 1000 mg.

The dose of the EGFR inhibitor of the present invention can be 1 mg, 2.5 mg, 5 mg, 7.5 mg, 10 mg, 12.5 mg, 15 mg, 17.5 mg, 20 mg, 22.5 mg, 25 mg, 27.5mg, 30 mg, 32.5 mg, 35 mg, 37.5 mg, 40 mg, 42.5 mg, 45 mg, 47.5 mg, 50 mg, 52.5 mg, 55 mg, 60 mg, 65 mg, 70 mg, 75 mg, 80 mg, 85 mg, 90 mg, 95 mg, 100 mg, 105 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg or 100 mg.

In the preferable embodiments of the present invention, the CDK4/6 inhibitor is administered once a day, and the dose thereof is preferably 25 mg, 50 mg, 75 mg, 100 mg, 125 mg, 150 mg or175 mg, the EGFR inhibitor is administered once a day, and the dose thereof is preferably 55 mg, 110 mg, 220 mg or260 mg.

In the preferable embodiments of the present invention, the CDK4/6 inhibitor is the compound represented by formula (I), the complex thereof or the pharmaceutically acceptable salt thereof, which is administered once a day, and the dose thereof is preferably 25 mg, 50 mg, 75 mg, 100 mg, 125 mg, 150 mg or 175 mg, the EGFR inhibitor is the compound represented by formula (II), the stereoisomer thereof, the complex thereof or the pharmaceutically acceptable salt thereof, which is administered once a day, and the dose thereof is preferably 55 mg, 110 mg, 220 mg or 260 mg.

In the optional embodiments of the present invention, the CDK4/6 inhibitor is the hydroxyethyl sulfonate of compound represented by formula (I), which is administered once a day, and the dose thereof can be 25 mg, 50 mg, 75 mg, 100 mg, 125 mg, 150 mg or 175 mg, the EGFR inhibitor is the mesylate of the compound represented by formula (II), which is administered once a day, and the dose thereof can be 55 mg, 110 mg, 220 mg or 260 mg.

In the preferable embodiments, the CDK4/6 inhibitor is the hydroxyethyl sulfonate of compound represented by formula (I), which is administered once a day, and the dose thereof is 25 mg, and the EGFR inhibitor is the mesylate of the compound represented by formula (II), which is administered once a day, and the dose thereof is preferably 55 mg, 110 mg, 220 mg or 260 mg.

In the preferable embodiments, the CDK4/6 inhibitor is the hydroxyethyl sulfonate of compound represented by formula (I), which is administered once a day, and the dose thereof is 50 mg, and the EGFR inhibitor is the mesylate of the compound represented by formula (II), which is administered once a day, and the dose thereof is preferably 55 mg, 110 mg, 220 mg or 260 mg.

In the preferable embodiments, the CDK4/6 inhibitor is the hydroxyethyl sulfonate of compound represented by formula (I), which is administered once a day, and the dose thereof is 75 mg, and the EGFR inhibitor is the mesylate of the compound represented by formula (II), which is administered once a day, and the dose thereof is preferably 55 mg, 110 mg, 220 mg or 260 mg.

In the preferable embodiments, the CDK4/6 inhibitor is the hydroxyethyl sulfonate of compound represented by formula (I), which is administered once a day, and the dose thereof is 100 mg, and the EGFR inhibitor is the mesylate of the compound represented by formula (II), which is administered once a day, and the dose thereof is preferably 55 mg, 110 mg, 220 mg or 260 mg.

In the preferable embodiments, the CDK4/6 inhibitor is the hydroxyethyl sulfonate of compound represented by formula (I), which is administered once a day, and the dose thereof is 125 mg, and the EGFR inhibitor is the mesylate of the compound represented by formula (II), which is administered once a day, and the dose thereof is preferably 55 mg, 110 mg, 220 mg or 260 mg.

In the preferable embodiments, the CDK4/6 inhibitor is the hydroxyethyl sulfonate of compound represented by formula (I), which is administered once a day, and the dose thereof is 150 mg, and the EGFR inhibitor is the mesylate of the compound represented by formula (II), which is administered once a day, and the dose thereof is preferably 55 mg, 110 mg, 220 mg or 260 mg.

In the preferable embodiments, the CDK4/6 inhibitor is the hydroxyethyl sulfonate of compound represented by formula (I), which is administered once a day, and the dose thereof is 175 mg, and the EGFR inhibitor is the mesylate of the compound represented by formula (II), which is administered once a day, and the dose thereof is preferably 55 mg, 110 mg, 220 mg or 260 mg.

In the preferable embodiments of the present invention, the weight ratio of the CDK4/6 inhibitor to the EGFR inhibitor is 0.001:1-1000:1, preferably, 0.01:1-100:1, the most preferably, 0.05:1-50:1, specifically can be 500:1, 400:1, 300:1, 200:1, 100:1, 50:1, 25:1, 12.5:1, 10:1, 8:1, 6:1, 5: 1, 3.18:1, 2.72:1, 2.27:1, 2:1, 1.81:1, 1.59:1, 1.36:1, 1.14:1, 1:1, 1:1.1, 1:1.5, 1:1.26, 1:1.47, 1:1.49, 1:1.73, 1:1.76, 1:2, 1:2.08, 1:2.2, 1:2.6, 1:2.93, 1:3.47, 1:3.5, 1:4.4, 1: 5, 1:5.2, 1:7.5, 1:8.8, 1:10, 1:10.4, 1:12.5, 1:15, 1:20, 1:25, 1:30, 1:50, 1:75, 1:100, 1:125, 1:150, 1:200, 1:250, 1:300, 1:400, 1:500, 1:600, 1:700 or 1:800.

The administration of the combination of the present invention can be oral administration, parenteral administration or transdermal administration, wherein the parenteral administration includes but not limited to intravenous injection, subcutaneous injection and intramuscular injection, preferably oral administration.

In the embodiments of the present invention, the combination optionally further contains other components, and the other components include but are not limited to other drugs for treating tumor diseases.

The present invention also provides a method for treating tumor disease, which comprises administering an effective amount of the CDK4/6 inhibitor and an effective amount of the EGFR inhibitor to a patient.

The present invention also provides a pharmaceutical composition, which comprises the CDK4/6 inhibitor, the EGFR inhibitor, and one or more than one pharmaceutical carriers, excipients or diluents. The pharmaceutical composition can be formulated as any pharmaceutically acceptable dosage form. For example, it can be formulated as tablets, capsules, pills, granules, solutions, suspensions, syrups, injections (including injection liquid, sterile powders for injection and concentrated solutions for injection), suppositories, inhalants or spray agents.

The pharmaceutical composition comprising the CDK4/6 inhibitor and the EGFR inhibitor of the present invention can be administered either alone or in combination with one or more than one therapeutic agents.

The present invention also provides a pharmaceutical kit which is in the use of the medicament for treating tumor disease, wherein the pharmaceutical composition comprising the CDK4/6 inhibitor and the EGFR inhibitor of the present invention is packaged.

In the present invention, the CDK4/6 inhibitor is administered in combination with the EGFR inhibitor, thereby enhancing the use of the medicament for tumor disease and improving the therapeutic effect.

The expression “in combination with” in the present invention is a mode of administration, which means that at least one dose of the CDK4/6 inhibitor and at least one dose of the EGFR inhibitor are administered within a certain period of time, wherein both of the two substances exhibit pharmacological effects. The period of time can be within one administration cycle, preferably within 4 weeks, within 3 weeks, within 2 weeks, within 1 week, or within 24 hours. The CDK4/6 inhibitor and the EGFR inhibitor can be administered simultaneously or sequentially. This period of time includes the treatment in which the CDK4/6 inhibitor and the EGFR inhibitor are administered via the same route or different routes.

The term “effective amount” refers to an amount of a drug effective to treat a disease or disorder in a mammal. In the case of cancer, the therapeutically effective amount of the drug may reduce the number of cancer cells, reduce the tumor size, inhibit (i.e., slow to some extent and preferably stop) cancer cell infiltration into peripheral organs, inhibit (i.e., slow to some extent and preferably stop) tumor metastasis, inhibit, to some extent, tumor growth, and/or relieve to some extent one or more than one symptoms associated with the disorder. Depending on the extent to which the drug may prevent the growth of and/or kill existing cancer cells, it may be cytostatic and/or cytotoxic. For cancer therapy, efficacy in vivo can, for example, be measured by assessing the duration of overall survival (OS), duration of progression free survival (PFS), the response rates (RR), duration of response, and/or quality of life.

### BRIEF DESCRIPTION OF THE DRAWINGS

FIG. 1 shows the effect of drug A in combination with drug B on the body weight of nude mice.

FIG. 2 shows the effect of drug A in combination with drug B on the tumor volume of the subcutaneously transplanted tumor model of human lung cancer cell NCI-H1975.

FIG. 3 shows the effect of drug A in combination with drug B on the relative tumor volume of the subcutaneously transplanted tumor model of human lung cancer cell NCI-H1975.

### DETAILED DESCRIPTION OF THE PREFERRED EMBODIMENT

Hereinafter, the present invention will be explained in more detail with reference to the embodiments. The embodiments of the present invention are only used to illustrate the technical solutions of the present invention, and do not limit the substantial and scope of the present invention.

Example 1 Evaluation of the hydroxyethyl sulfonate of the compound represented by formula (I) (drug A) in combination with the mesylate of compound represented by formula (II) (drug B) on the proliferation inhibition of human non-small cell lung adenocarcinoma cells (NCI-H1975).

1. Experimental Materials

Human lung adenocarcinoma cells NCI-H1975 (carrying the EGFR21 exon L858R mutation and the 20 exon 1790M mutation) were incubated by the Cancer and Endocrine Pharmacology Laboratory, Department of Pharmacy, Zhejiang University and stored in liquid nitrogen;

Drug A was prepared according to the method disclosed in WO2016124067A;

Drug B was prepared according to the method disclosed in WO2017161937A.

2. Experimental Method

NCI-H1975 cells with good growth status were seeded in 96-well plates, and after overnight adherent growth, they were administrated with test substances in different concentrations respectively, in triple replicate for each concentration. The final concentration of the test substance was set to 0.125, 0.25, 0.5, 1 and 2 μM for the hydroxyethyl sulfonate of the compound represented by formula (I), and was set to 5 and 10 nM for the compound represented by formula (II). After NCI-H1975 cells were treated with the test substances alone or in combination for 7 days, the in vitro proliferation inhibitory effect of the test substances on NCI-H1975 cells was detected based on the SRB method.

3. Results Processing

According to the OD value measured by the microplate reader, the inhibition rate was calculated based on the following formula:

Inhibition rate (%)=(1−OD_{administration}/OD_{control})×100%

4. Experimental Results

The hydroxyethyl sulfonate of the compound represented by formula (I) can inhibit the growth of NCI-H1975 cells in a concentration-dependent manner within a concentration range of 0.125-2 μM. Drug A in combination with 5 nM or 10 nM drug B can increase the proliferation inhibition rate of NCI-H1975 cells relative to the two drugs used alone. See Table 1 for details.

TABLE 1

| Drug A | | Drug B | | In Combination |
|---|---|---|---|---|
| Concentration | Inhibition | Concentration | Inhibition | Inhibition |
| (μM) | rate (%) | (nM) | rate (%) | rate (%) |
| 0.125 | 46.3 | 5 | 50.3 | 77.3 |
| 0.25 | 50.0 | | | 81.3 |
| 0.5 | 56.0 | | | 84.3 |
| 1 | 62.4 | | | 85.5 |
| 2 | 73.5 | | | 90.6 |
| 0.125 | 46.3 | 10 | 53.8 | 82.2 |
| 0.25 | 50.0 | | | 82.0 |
| 0.5 | 56.0 | | | 83.6 |
| 1 | 62.4 | | | 85.0 |
| 2 | 73.5 | | | 92.3 |

Example 2 Evaluation of the experimental therapeutic effect of drug A in combination with drug B on human lung cancer NCI-H1975 transplanted tumor model of nude mice

1. Experimental Materials

Experimental animals: nude mice, female; 8 weeks, 24 vaccinated, 18 actually used, provided by Shanghai Xipuer-Bikai Experimental Animal Co., Ltd.;

Test drugs: Drug A and Drug B are the same as those used in Example 1, the control drug gefitinib was provided by the Department of Pharmacy, Zhejiang University.

2. Experimental Method

1) Dose and Frequency of Administration

Gefitinib: 30 mg/kg, once a day, Drug A: 100 (50) mg/kg, once a day, Drug B: 5 mg/kg, once a day.

2) Drug Formulation

6 mg gefitinib was weighed, added with an appropriate amount of 0.5% CMC-Na, grinded evenly, transferred to an EP tube, added with 0.5% CMC-Na to 2 mL, and mixed evenly to obtain a solution of gefitinib with a concentration of 3 mg/mL, which was freshly prepared before use.

20 mg drug A was weighed, added with an appropriate amount of 0.5% MC, grinded evenly, transferred to an EP tube, added with 0.5% MC to 2 mL, and mixed evenly to obtain a solution of drug A with a concentration of 10 mg/mL, which was freshly prepared before use.

1 mg drug B was weighed, added with an appropriate amount of 0.5% MC, grinded evenly, transferred to an EP tube, added with 0.5% MC to 2 mL, and mixed evenly to obtain a solution of drug B with a concentration of 0.5 mg/mL, which was freshly prepared before use.

3) Model Establishment

1×10⁷ human lung cancer cells NCI-H1975 cells were injected into the armpits of nude mice. After the tumor grew to a suitable size, the NCI-H1975 mouse tumor was removed and placed in a container filled with saline. The surface blood vessels were peeled off and the necrotic area was discarded. The tumor was then cut into 1-2 mm³, and the tumor was inserted into the left armpit of the nude mouse with a trocar. After the tumor grew to an average volume of 50-150 mm³, 18 mice were divided into 6 groups (3 in each group), namely the negative control group (Control), the group of “gefitinib at a dose of 30 mg/kg”, the group of “drug A at a dose of 100(50) mg/kg”, the group of “drug B at a dose of 5 mg/kg”, the group of “drug A in combination with drug B” and the group of “drug A in combination with gefitinib”.

4) Specific Procedure

All nude mice were administered by intragastric administration, the administration volume was 10 mL/kg, and the tumor volume was weighed and measured twice a week. The administration cycle was 21 days. On the 22^{nd} day, the mice were weighed and the tumor volume was measured. The mice were then sacrificed and the tumor was weighed. The relative tumor volume (RTV), relative tumor growth rate (T/C) and tumor inhibition percentage (IR) were calculated and the statistical test (SPSS test) was performed.

Calculation formula shown as follows:

(1) TV (tumor volume)=½×a×b², wherein, a and b represent the length and width of the tumor, respectively;

(2) RTV (relative tumor volume)=Vₜ/V₀, wherein, V₀ is the tumor volume measured at grouping (i.e., d₀), and Vₜ is the tumor volume at each measurement;

(3) T/C(%)=T_{RTV}/C_{RTV}×100%, wherein T_{RTV} is the RTV of the treatment group, and C_{RTV} is the RTV of the control group;

(4) IR (%)=(1−TWₜ/TW_{c})×100%, wherein TWₜ is the tumor weight of the treatment group, TW_{c} is the tumor weight of the control group.

3. Experimental Results

At the end of the experiment, compared with the negative control group, the body weight of mice in each administration group has no significant change. (See Table 2 and FIG. 1 for details).

TABLE 2
The effect of drug A in combination with drug B on the
body weight of nude mice (x̅ ± SE)

| | | | Animal | | Average body | |
|---|---|---|---|---|---|---|
| | | Dose | number | | weight (g) | |
| Group | Drug | (mg/kg) | D 1 | D 22 | D 1 | D 22 |
| 1 | Negative | — | 3 | 3 | 22.7 ± 0.3 | 24.7 ± 0.4 |
| | control | | | | | |
| 2 | Gefitinib | 30 | 3 | 3 | 21.8 ± 0.6 | 24.5 ± 0.8 |
| 3 | Drug A | 100(50) | 3 | 3 | 22.8 ± 0.3 | 23.5 ± 0.7 |
| 4 | Drug B | 5 | 3 | 3 | 22.8 ± 0.7 | 21.8 ± 1.4 |
| 5 | Drug A + | 100(50) + 5 | 3 | 3 | 22.3 ± 0.4 | 20.4 ± 0.9 |
| | Drug B | | | | | |
| 6 | Drug A + | 100(50) + 30 | 3 | 3 | 20.7 ± 0.6 | 22.5 ± 0.8 |
| | Gefitinib | | | | | |

From Day 1 to Day 12, the dose of drug A in the group of “drug A” and the group of “combination” was 100 mg/kg. From Day 13 to Day 21, the dose was changed to 50 mg/kg. Compared with the negative control, there is no significant difference in each group.

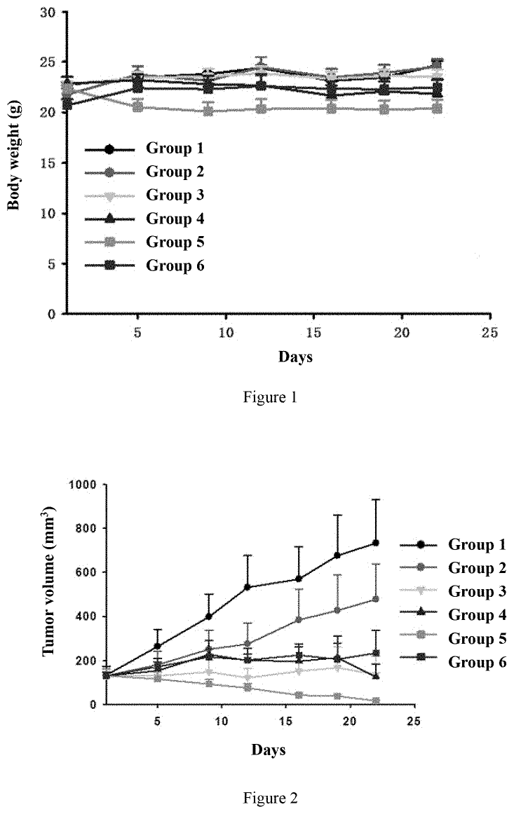

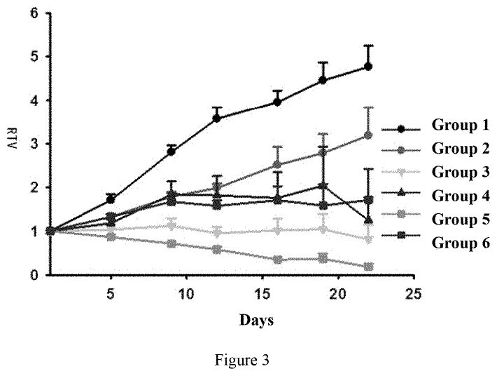

At the end of the experiment, compared with the tumor volume of the negative control group being 732±200 mm³, the tumor volumes of group 2 to group 6 are 477±162 mm³, 136±83 mm³, 125±58 mm³, 17±3 mm³ and 233±104 mm³, respectively (see Table 3 and FIG. 2 for details). Compared with the RTV value of the negative control group being 4.77±0.48, the RTV values of group 2 to group 6 are 3.18±0.65, 0.81±0.33, 1.25±0.53, 0.18±0.07 and 1.71±0.71, respectively; and T/C values are 66.81%, 17.00%, 26.14%, 3.86% and 35.91%, respectively (see Table 3 and FIG. 3 for details).

TABLE 3
Effect of drug A in combination with drug B on subcutaneous
transplanted tumor (x̅ ± SE)

Tumor volume (mm³)

| Group | D 1 | D 22 | RTV | T/C(%) |
|---|---|---|---|---|
| 1 | 133 ± 35 | 732 ± 200 | 4.77 ± 0.48 | — |
| 2 | 130 ± 41 | 477 ± 162 | 3.18 ± 0.65 | 66.81 |
| 3 | 130 ± 36 | 136 ± 83 | 0.81 ± 0.33 | 17.00 |
| 4 | 130 ± 44 | 125 ± 58 | 1.25 ± 0.53 | 26.14 |
| 5 | 130 ± 29 | 17 ± 3 | 0.18 ± 0.07 | 3.86 |
| 6 | 130 ± 17 | 233 ± 104 | 1.71 ± 0.71 | 35.91 |

At the end of the experiment, compared with the tumor weight of the the negative control group being 0.5958±0.1900 g, the tumor weights of group 2 to group 6 are 0.4097±0.1605 g, 0.1269±0.0839 g, 0.1091±0.0621 g, 0.0061±0.0002 g and 0.2153±0.1119 g, respectively; and IR are 31.24%, 78.69%, 81.69%, 98.98% and 63.86%, respectively (see Table 4 for details).

TABLE 4
Effect of drug A in combination with drug
B on the weight of subcutaneous transplanted
tumor (x̅ ± SE)

| Group | Tumor weight (g) | IR (%) |
|---|---|---|
| 1 | 0.5958 ± 0.1900 | — |
| 2 | 0.4097 ± 0.1605 | 31.24 |
| 3 | 0.1269 ± 0.0839 | 78.69 |
| 4 | 0.1091 ± 0.0621 | 81.69 |
| 5 | 0.0061 ± 0.0002 | 98.98 |
| 6 | 0.2153 ± 0.1119 | 63.86 |

From Day 1 to Day 12, the dose of drug A in the group of “drug A” and the group of “combination” was 100 mg/kg. From Day 13 to Day 21, the dose was changed to 50 mg/kg.

Under the experimental conditions, drug A at a dose of 100 (50) mg/kg in combination with drug B at a dose of 5 mg/kg (q.d., 21 days in total) can inhibit the growth of human lung cancer NCI-H1975 transplanted tumors in nude mice, and the effect is better than that of drug A in combination with gefitinib.

1. A method for preventing or treating tumor disease, comprising administering to a subject in need thereof an effective amount of a CDK4/6 inhibitor and an EGFR inhibitor.

2. The method as defined in claim 1, wherein the tumor disease is selected from the group consisting of breast cancer, ovarian cancer, prostate cancer, melanoma, brain tumor, esophageal cancer, stomach cancer, liver cancer, pancreatic cancer, colorectal cancer, lung cancer, kidney cancer, skin cancer, glioblastoma, neuroblastoma, sarcoma, liposarcoma, osteochondroma, osteoma, osteosarcoma, seminoma, testicular tumor, uterine cancer, head and neck tumor, multiple myeloma, malignant lymphoma, polycythemia vera, leukemia, thyroid tumor, ureteral tumor, bladder tumor, gallbladder cancer, cholangiocarcinoma or choriocarcinoma; preferably, non-small cell lung cancer.

3. The method as defined in claim 1, wherein the tumor disease is an EGFR mutation tumor disease.

4. The method as defined in claim 3, wherein the EGFR mutation tumor disease is non-small cell lung cancer, the EGFR mutant is preferably L858R EGFR mutant and/or T790M EGFR mutant.

5. The method as defined in claim 2, wherein the non-small cell lung cancer is squamous cell carcinoma or non-squamous cell carcinoma, preferably, non-phosphorous cell carcinoma.

6. The method as defined in claim 1, wherein the CDK4/6 inhibitor is selected from the group consisting of abemaciclib, ribociclib, palbociclib, alvocidib, trilaciclib, voruciclib, AT-7519, G1T-38, FLX-925, INOC-005, G1T28-1, BPI-1178, gossypin, G1T30-1, GZ-38-1, P-276-00, staurosporine, R-547, PAN-1215, PD-0183812, AG-024322, NSC-625987, CGP-82996, PD-171851 or the compound represented by formula (I), the complex thereof and the pharmaceutically acceptable salt thereof, preferably abemaciclib, ribociclib, palbociclib, alvocidib, the compound represented by formula (I), the complex thereof and the pharmaceutically acceptable salt thereof, the most preferably the compound represented by formula (I), the complex thereof and the pharmaceutically acceptable salt thereof,

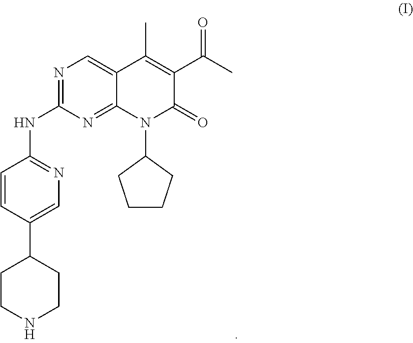 [SMILES: CC(=O)c1c(C)c2cnc(Nc3ccc(C4CCNCC4)cn3)nc2n(C2CCCC2)c1=O]

7. The method as defined in claim 1, wherein the EGFR inhibitor is selected from the group consisting of osimertinib, gefitinib, erlotinib, olmutinib, icotinib, pyrotinib, vandetanib, brigatinib, dacomitinib, afatinib, neratinib, lapatinib, ABT-414, varlitinib, HLX-07, tesevatinib, theliatinib, epitinib succinate, S-222611, poziotinib, AST-2818, GNS-1480, mavelertinib, AP-32788, AZD-3759, nazartinib, Sym-013, allitinib tosylate, tarloxotinib bromide, CK-101, QL-1203, JNJ-61186372, SKLB-1028, TAS-121, Hemay-020, Hemay-022, NRC-2694-A, simotinib hydrochloride, SPH-1188-11, GR-1401, SYN-004, ABBV-221, MP-0274, GC-1118, BPI-15000, DBPR-112, Pirotinib, PB-357, lifirafenib, SCT-200, QLNC-120, agerafenib hydrochloride, the compound represented by formula (II), the stereoisomer thereof, the complex thereof and the pharmaceutically acceptable salt thereof, preferably olmutinib, afatinib, osimertinib, CK-101, erlotinib, icotinib, gefitinib, the compound represented by formula (II), the stereoisomer thereof, the complex thereof and the pharmaceutically acceptable salt thereof, the most preferably the compound represented by formula (II), the stereoisomer thereof, the complex thereof and the pharmaceutically acceptable salt thereof,

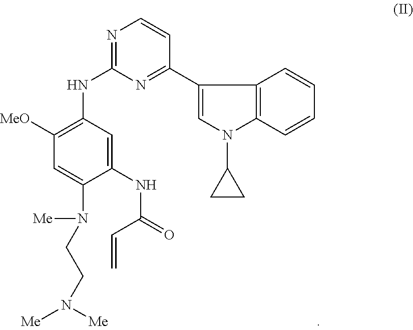 [SMILES: C=CC(=O)Nc1cc(Nc2nccc(-c3cn(C4CC4)c4ccccc34)n2)c(OC)cc1N(C)CCN(C)C]

8. The method as defined in claim 6, wherein the pharmaceutically acceptable salt of the compound represented by represented by formula (I) is hydroxyethyl sulfonate.

9. The method as defined in claim 7, wherein the pharmaceutically acceptable salt of the compound represented by represented by formula (II) is mesylate.

10. The method as defined in claim 1, wherein the dose of the CDK4/6 inhibitor is 1-1000 mg, and the frequency of administration thereof is once a day, twice a day, or three times a day, and the dose of the EGFR inhibitor is 1-1000 mg, and the frequency of administration thereof is once a day, twice a day, or three times a day.

11. The method as defined in claim 10, wherein the weight ratio of the CDK4/6 inhibitor to the EGFR inhibitor is 0.001:1-1000:1, preferably 0.01:1-100:1, the most preferably 0.05:1-50:1.

12. The method as defined in claim 10, wherein the CDK4/6 inhibitor is administered once a day, and the dose thereof is 25 mg, 50 mg, 75 mg, 100 mg, 125 mg, 150 mg and 175 mg, and the EGFR inhibitor is administered once a day, and the dose thereof is 55 mg, 110 mg, 220 mg and 260 mg.

13. A pharmaceutical composition, which comprises a CDK4/6 inhibitor and an EGFR inhibitor, and one or more pharmaceutical carriers, excipients or diluents.

14. A pharmaceutical kit, which comprises the pharmaceutical composition as defined in claim 13.
